(19) United States
(12) Patent Application Publication
(10) Pub. No.: US 2022/0387514 A1
(43) Pub. Date: Dec. 8, 2022

(54) METHODS OF TREATING ACUTE RESPIRATORY DISTRESS SYNDROME

(71) Applicant: Kadimastem Ltd., Nes Ziona (IL)

(72) Inventors: Michel REVEL, Rehovot (IL); Michal IZRAEL, Tel-Aviv (IL); Julia Miriam WEISS, Rehovot (IL); Kfir MOLAKANDOV, Yehud-Monosson (IL)

(73) Assignee: Kadimastem Ltd., Nes Ziona (IL)

(21) Appl. No.: 17/830,425

(22) Filed: Jun. 2, 2022

Related U.S. Application Data
(60) Provisional application No. 63/195,768, filed on Jun. 2, 2021.

(51) Int. Cl. A61K 35/30; A61K 35/28; A61P 11/00; A61P 37/06

(52) U.S. Cl. CPC A61K 35/30; A61K 35/28; A61P 11/00; A61P 37/06

(57) ABSTRACT

A method of treating acute respiratory distress syndrome (ARDS) in a subject in need thereof is provided. The method comprising administering to the subject a composition comprising a therapeutically effective amount of astrocytes, thereby treating the ARDS.

### RELATED APPLICATION(S)

This application claims the benefit of priority under 35 USC 119(e) of U.S. Provisional Patent Application No. 63/195,768 filed on Jun. 2, 2021, the contents of which are all incorporated by reference as if fully set forth herein in their entirety.

### FIELD AND BACKGROUND OF THE INVENTION

The present invention, in some embodiments thereof, relates to methods of treating acute respiratory distress syndrome.

Acute respiratory distress syndrome (ARDS) is a form of progressive hypoxemia respiratory failure and pulmonary edema in the absence of heart failure 1. The etiology of ARDS varies (e.g. pneumonia, sepsis, or acute pancreatitis) and great efforts have been made in intensive care medicine, but the overall mortality is still high^{2, 3}. A significant proportion of the causative agent of coronavirus disease 2019 (COVID-19) infected patients develop viral pneumonia that causes an acute lung injury capable of rapid progression to viral sepsis and ARDS with a high fatality rate especially in older and comorbidities populations^{4, 5, 6}. The phases in the development of ARDS includes exudation stage characterized by inflammatory cell infiltration and pulmonary edema (stage 1), the proliferation of myofibroblasts (stage 2) and extracellular matrix (ECM) over-deposited (stage 3) 7. The fibrosis process (stage 2 and 3) is rapid and occurs within one week 8, 9. This inflammatory response process, also referred to as cytokine storm or cytokine release syndrome (CRS), contributes to the development of ARDS and often irreversible multi-organ dysfunction syndrome (MODS) associated with the severe-critical forms of COVID-19 10, 11. Another key process in the development of ARDS is neutrophil accumulation in high abundance in the pulmonary microcirculation, lung interstitium and alveolar airspace of ARDS patients 12. ARDS is also associated with systemic neutrophil priming, delayed neutrophil apoptosis and clearance of neutrophils from the lungs. In animal models, lung injury could be ameliorated by reducing the number of circulating neutrophil-13.

Treatment platforms capable of immunomodulating the immune system and inflammatory response could potentially be of great benefit in preventing the disease progression and reducing the case mortality rate in high-risk COVID-19 patients.

Astrocytes are the most abundant glial cell in the central nervous system. Astrocytes regulate the concentration of different neurotransmitters and ions, supply various metabolites and energy, regulate osmolarity, modulate synaptic activity, secrete neurotrophic and neuroprotective factors, promote neurogenesis^{14, 15, 16} and remyelination¹⁷. Moreover, Astrocytes are essential players in immune-modulation^{18 19}. Astrocytes have also emerged as key contributors to the innate immune response of the CNS to infections, neurodegenerative disorders, and injuries²⁰. Following injury and in disease, their ability to respond to, and commence initial responses to injury/disease is increasingly apparent, as such, astrocytes serve as contact between the CNS and the peripheral immune system. In many pathological conditions, astrocytes may secrete anti-inflammatory and pro-inflammatory factors which modulate the immune system^{21 22-23}. Astrocytes, thus, take part in both the recruitment and restriction of leukocytes in the CNS^{24, 25}.

The immune-modulatory effect of astrocytes outside the CNS is still not well characterized, especially of ex-vivo differentiated astrocytes.

Additional background art includes:

Recent reports suggest that COVID19 is associated with damage to the central nervous system (e.g., Ladecola et al. Cell 183, Oct. 1, 2020 ^{a} 2020 Elsevier Inc.; Murta et al. doi:10.20944/preprints202006.0319.v1).

Intratracheal administration of mesenchymal stem cells secreting neurotrophic factors (MSC-NTF)-derived exosomes provided a statistically significant reduction in lung disease severity score as reported in https://www(dot)clinicaltrialsarena(dot)com/news/brainstorm-treatment-preclinical-study/

### SUMMARY OF THE INVENTION

According to an aspect of some embodiments of the present invention there is provided a method of treating acute respiratory distress syndrome (ARDS) in a subject in need thereof, the method comprising administering to the subject a composition comprising a therapeutically effective amount of astrocytes, thereby treating the ARDS.

According to an aspect of some embodiments of the present invention there is provided a composition comprising a therapeutically effective amount of astrocytes for use in treating acute respiratory distress syndrome (ARDS).

According to some embodiments of the invention, the astrocytes having been ex vivo differentiated from stem cells.

According to some embodiments of the invention, the stem cells are pluripotent stem cells.

According to some embodiments of the invention, the pluripotent stem cells are embryonic stem cells.

According to some embodiments of the invention, the pluripotent stem cells are induced pluripotent stem (IPS) cells.

According to some embodiments of the invention, the stem cells are mesenchymal stem cells.

According to some embodiments of the invention, the method further comprises ex vivo differentiating the astrocytes prior to the administering. According to some embodiments of the invention, the ex vivo differentiating the astrocytes from the pluripotent stem cells comprise:

(a) producing neurospheres (NS) from the pluripotent stem cells in a suspension culture in the presence of EGF and retinoic acid;
(b) dissociating the NS so as to obtain astrocyte progenitor cells (APCs);
(c) expanding the APCs in an adherent culture in the presence of EGF and bFGF;
(d) differentiating the APCs to committed astrocytes in the presence of ascorbic acid.

According to some embodiments of the invention, the astrocytes are formulated for intravenous or intratracheal administration. According to some embodiments of the invention, the ARDS is associated with virus infection.

According to some embodiments of the invention, the virus is a Coronavirus.

According to some embodiments of the invention, the Coronavirus is SARS-CoV-2.

According to some embodiments of the invention, the astrocytes reduce neutrophil accumulation at the site of inflammation associated with the ARDS.

According to some embodiments of the invention, the astrocytes reduce pro-inflammatory cytokines.

According to some embodiments of the invention, the pro-inflammatory cytokines are selected from the group consisting of TNF-alpha, ILb1 and IL-6.

According to some embodiments of the invention, the astrocytes reduce T cells proliferation.

According to some embodiments of the invention, the astrocytes reduce fibrosis.

According to some embodiments of the invention, the composition comprises a viable form of the astrocytes in predominant manner.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show that ex vivo differentiated human astrocytes (AstroRx®) demonstrate immunomodulatory capacity in-vitro. A. Flow Cytometry analysis of astrocytic markers of AstroRx®. B. Flow Cytometry analysis of pluripotent stem cells markers in AstroRx®. C and D. Immunosuppressive potential of AstroRx® was assessed by mixed lymphocyte reaction test. AstroRx® were co-cultured with murine lymph node cells (LNC). C. Assessment of the effect of AstroRx® on the percentage of Thy1.2 and CD25 positive cells under ConA activation, D. The proliferation state of T cells (CD3+) was determined by proliferation marker BrdU in the presence of ConA. LNC only was used as negative control. Average and t SD.

FIGS. 2A-D show that AstroRx cells alleviate LPS-induced pulmonary inflammation and fibrosis. A. Survival rate of animals treated with Plasmalyte (sham) and AstroRx cells (500K per animal). B. Hematoxylin and eosin (H&E) staining of lung sections from top to bottom: LPS-induced treated with vehicle (PlasmaLyte), LPS-induced treated with 500K AstroRx cells, or naive, healthy controls. C. A quantitative analysis for Acute Lung Injury (ALI) was performed using a severity scoring scale of 0-2 (20 fields per animal were analyzed), based on the American Thoracic Society Documents, 2011 (Matute-Bello et al., Am J Respir Cell Mol Biol 44; 725-738, 2011). D. Percentage of responder mice defined by a lung score ≤2 taking into account also the mice that had died from ARDS. n=10, results are expressed as meant SEM. Mann-Whitney comparison test. *p<0.05; **p<0.01; ***p<0.001. Scale bar: 50 μM.

FIGS. 3A-B show immune cells analyses of Bronchoalveolar lavage. Bronchoalveolar lavage (BALF) was analyzed by flow cytometry for T cells, B cells, eosinophils, neutrophils and macrophages/dendritic cells. A. Proportions of each cell population by treatment. B. Absolute number of T cells, Eosinophils, Macrophages and dendritic cells (DC), B cells and Neutrophils in the Bronchoalveolar lavage fluid (BALF). Results are expressed as mean t SEM. Mann-Whitney comparison test. *p<0.05; **p<0.01.

FIG. 4 shows that CXCL1 and TNFa levels in the peripheral blood of AstroRx-cell treated mice mirror the situation in the lungs. CXCL1 and TNFa levels in the blood serum were quantified by ELISA. n=10, results are expressed as mean t SEM. Mann Whitney comparison test. *p<0.05.

### DESCRIPTION OF SPECIFIC EMBODIMENTS OF THE INVENTION

The present invention, in some embodiments thereof, relates to methods of treating acute respiratory distress syndrome.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Acute respiratory distress syndrome (ARDS) is caused by increased amounts of pro-inflammatory cytokines and neutrophil-mediated tissue injury. To date, there is no effective therapies for treating ARDS, with emerging need due to most severe complications of the current COVID-19 pandemic.

Whilst conceiving and reducing to practice embodiments of the invention, the present inventors envisaged that astrocytes can serve as a potential treatment for ARDS.

First the ability of clinical grade human astrocytes (which are termed according to some embodiments “AstroRx™”) to ameliorate T cell proliferation was assessed using mixed lymphocyte reaction test. Next, the capability of AstroRx™ cells to treat ARDS was tested by intravenous (i.v.) injection of AstroRx™ in LPS-induced lung injury mouse model. Measurements of lung histopathological changes were analyzed by scoring the presence of Neutrophils and Fibrin as well as measuring thickened alveolar walls. In addition, immune-cell profile as well as proinflammatory cytokines and chemokines were also assessed in bronchoalveolar lavage fluid (BALF) and serum.

It was found that ex-vivo differentiated astrocytes (AstroRx™) suppressed T cell proliferation in-vitro in response to the mitogen Concanavalin A. In addition, ex-vivo differentiated astrocytes-treated mice had higher survival rate in LPS-induced ARDS animal model. Moderate lung injury was observed in ex-vivo differentiated astrocytes treated animals compared to severe lung injury in Sham injected animals. Astrocyte-treated mice presented a steady number of macrophages/DCs, T cells and neutrophils. Inflammatory cytokines TNFα and CXCL1 chemokine in the serum were lower in r ex-vivo differentiated astrocytes-treated mice compared to sham injected mice.

Altogether, these results demonstrate the immunomodulatory activity of AstroRx™ cells in peripheral inflammation. These positive preclinical results support the potential of astrocytes as a therapy for the treatment of ARDS.

Thus, according to an aspect of the invention there is provided a method of treating acute respiratory distress syndrome (ARDS) in a subject in need thereof, the method comprising administering to the subject a composition comprising a therapeutically effective amount of astrocytes, thereby treating ARDS.

Alternatively or additionally, there is provided a composition comprising a therapeutically effective amount of astrocytes for use in treating acute respiratory distress syndrome (ARDS).

As used herein, the term “treating” includes abrogating, substantially inhibiting, slowing or reversing the progression of a condition, substantially ameliorating clinical or aesthetical symptoms of a condition.

According to a specific embodiment, “treating” also includes substantially preventing the appearance of clinical or aesthetical symptoms of a condition.

According to specific embodiments, the ARDS is not in co-morbidity with other inflammatory diseases as those of the central nervous system, such as motor neuron diseases, progressive muscular atrophy (PMA), spinal muscular atrophy (SMA), progressive bulbar palsy, pseudobulbar palsy, primary lateral sclerosis, neurological consequences of AIDS, amyotrophic lateral sclerosis (ALS), Alzheimer's disease, developmental disorders, epilepsy, multiple sclerosis, neurogenetic disorders, Parkinson's disease, neurodegenerative disorders, stroke, spinal cord injury and traumatic brain injury.

Acute Respiratory Distress Syndrome (ARDS) is a respiratory failure characterized by rapid onset of widespread inflammation in the lungs. Symptoms include shortness of breath, rapid breathing, and bluish skin coloration. The underlying mechanism involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide.

Methods of diagnosing ARDS are known in the art and include a PaO₂/FiO₂ ratio (ratio of partial pressure arterial oxygen and fraction of inspired oxygen) of less than 300 mm Hg despite a positive end-expiratory pressure (PEEP) of more than 5 cm H₂O.

Viral infection, sepsis, pancreatitis, trauma, pneumonia, and aspiration are non-limiting examples of underlying causes of ARDS.

According to specific embodiments, the ARDS is associated with an infectious disease.

As used herein, the term “associated with infectious disease” means that a pathogen infection leads to the ARDS.

According to specific embodiments, the disease is an infectious disease.

As used herein, the term “infection” or “infectious disease” refers to a disease induced by a pathogen. Non-limiting specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), intracellular protozoan pathogens (such as, for example, Leishmania and Trypanosoma), parasitic diseases, fungal diseases, prion diseases.

Methods of analyzing infections are well known in the art and are typically based on serology, protein markers, or nucleic acid assays.

According to specific embodiments, the infectious disease is associated with a viral infection.

As used herein, the term “associated with a viral infection” means that a viral infection leads to the disease.

Specific types of viral pathogens causing infectious diseases treatable according to specific embodiments of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Non-limiting examples of viral infections include human immunodeficiency virus (HIV)-induced acquired immunodeficiency syndrome (AIDS), coronavirus, influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the ARDS is associated with a viral infection.

According to specific embodiments, the viral infection is a respiratory viral infection.

Non-limiting examples of respiratory viral infections associated with ARDS include a Coronavirus infection, a respiratory syncytial virus (RSV) infection, an influenza virus infection, a parainfluenza virus infection, an adenovirus infection and a rhinovirus infection.

According to specific embodiments, the viral infection is a Coronavirus infection.

According to specific embodiments, a clinical manifestation of Coronavirus infection includes symptoms selected from the group consisting of inflammation in the lung, alveolar damage, ARDS, fever, cough, shortness of breath, diarrhea, organ failure, pneumonia, cytokine storm, septic shock and/or blood clots.

As used herein, “Coronavirus” refers to enveloped positive-stranded RNA viruses that belong to the family Coronaviridae and the order Nidovirales.

Examples of Coronaviruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Coronaviruses, which are zoonotic in origin, can evolve into a strain that can infect human beings leading to fatal illness. Thus, particular examples of Coronaviruses contemplated herein are SARS-CoV, Middle East respiratory syndrome Coronavirus (MERS-CoV), and the recently identified SAR-CoV-2 [causing 2019-nCoV (also referred to as “COVID-19”)].

It would be appreciated that any Coronavirus strain is contemplated herein even though SAR-CoV-2 is emphasized in a detailed manner.

According to specific embodiments, the Coronavirus is SAR-CoV-2.

As used herein the phrase “subject in need thereof” refers to a mammalian male or female subject (e.g., human being).

According to some embodiments, the subject is under 18 years old.

According to some embodiments, the subject is 18-59 years old.

According to some embodiments, the subject is over 60 years old.

According to specific embodiments, the subject is diagnosed with a pathology (e.g. ARDS, infectious disease e.g. Coronavirus infection).

According to specific embodiments, the subject is at risk of developing a pathology (e.g. ARDS, infectious disease e.g. Coronavirus infection).

According to some embodiments, the subject is immunodeficient.

According to specific embodiments, the subject does not have an inflammatory disease other than the ARDS or infectious disease (e.g. Coronavirus infection).

The term “cytokine storm syndrome”, also referred to as “cytokine storm”, “cytokine release syndrome” or “inflammatory cascade”, as used herein refers to the systemic inflammatory condition involving elevated levels of circulating cytokines, causing immune-cell hyperactivation, and typically leading to multisystem organ dysfunction and/or failure which can lead to death. Often, a cytokine storm is referred to as being part of a sequence or cascade because one pro-inflammatory cytokine typically leads to the production of multiple other pro-inflammatory cytokines that can reinforce and amplify the immune response.

Diagnosis of cytokine storm syndrome can be carried out using any method known in the art, such as by a subject's physical evaluation, blood tests and imaging-based evaluation. Early symptoms of cytokine storm may include, for example, high fever, fatigue, anorexia, headache, rash, diarrhea, arthralgia, myalgia, and neuropsychiatric symptoms, or any combination thereof. However, early symptoms may quickly (e.g. within hours or within days) turn into more severe and life-threating symptoms. Accordingly, subjects having cytokine storm syndrome typically have respiratory symptoms, including cough and tachypnea that can progress to acute respiratory distress syndrome (ARDS), with hypoxemia that may require mechanical ventilation. Severe symptoms of cytokine storm may include, for example, uncontrollable hemorrhaging, severe metabolism dysregulation, hypotension, cardiomyopathy, tachycardia, dyspnea, fever, ischemia or insufficient tissue perfusion, kidney failure, liver injury acute liver injury or cholestasis, multisystem organ failure, or any combination thereof. Blood tests typically illustrate hyperinflammation as measured, for example, by C-reactive protein (CRP) levels, and blood-count abnormalities, such as leukocytosis, leukopenia, anemia, thrombocytopenia, and elevated ferritin and d-dimer levels.

According to one embodiment, cytokine storm syndrome is typically associated with elevated serum levels of at least 10%, at least 20%, at least 30% at least 40%, at least 50%, at least 60%, at least 70%, e.g. at least 50% (compared to basal state) of one or more cytokine, such as but not limited to, IFN-α, IFN-7, TNF-α, IL-1 (e.g. IL-1a, IL-1p), IL-2, IL-5, IL-6, IL-7, IL-12, IL-178, IL-18, IL-21, IL-17, IL-33 and HMGB-1, or chemokine, such as but not limited to, IL-8, MIG, IP-10, MCP-1 (e.g., MIP-1α, MIP-1β), and BLC. Assessment of cytokine levels can be carried out using any method known in the art, such as but not limited to, by ELISA or immunoassay.

According to a specific embodiment, the cytokine is selected from the group consisting of IL-5, MCP-1/CCL-2 and CXCL-1.

According to a specific embodiment, the cytokine is IL-5.

According to one embodiment, the subject may be a subject at any stage of the cytokine storm, e.g. a subject showing preliminary signs of a cytokine storm (e.g. elevated CRP levels, elevated cytokine levels, having early symptoms of cytokine storm as discussed above), a subject showing mild signs of cytokine storm (e.g. showing signs of organ dysfunction, requiring oxygen, blood tests showing hyperinflammation), a subject having severe signs of cytokine storm (e.g. requiring mechanical ventilation, hemorrhaging, having multisystem organ dysfunction and/or failure) or a subject after the severe stage of a cytokine storm.

According to a specific embodiment, the subject is at risk of developing a cytokine storm.

According to a specific embodiment, the ARDS is associated with neutrophil accumulation or infiltration to the lungs, as reviewed extensively in Rebetz et al. Transfus Med Hemother 2018; 45:290-298.

Neutrophils (PMNs) are derived from hematopoietic stem cells in the bone marrow and are approximately 12-14 μm in diameter with a multilobed nucleus and a granular cytoplasm. They are the most common type of granulocytes and constitute more than half of all circulating leukocytes. In healthy adults, around 36% of all PMNs are residing in the circulation, and of the total number of PMNs around 28%, both circulating and non-circulating, are suggested to be present in the pulmonary pool. The number of PMNs in the pulmonary pool is subject to change upon systemic inflammatory conditions. During inflammation, PMNs are the first responders and recruited in large numbers to the inflammatory microenvironment by the accumulation of lipid mediators, cytokines, and chemokines as well as changes to the vascular endothelium. The migration of PMNs towards the inflamed tissue is a complex interplay between the PMN and the adhesive molecules on the vascular endothelium. This multi-step process involves PMN-endothelial tethering, rolling, adhesion, crawling, and PMN-transendothelial migration. The early tethering stage involves cell adhesion molecules, e.g. selectins, on the endothelial cells and their ligands such as P-selectin glycoprotein ligand 1 (PSGL1) on PMNs. During the subsequent rolling step, chemokine receptors are engaged, and neutrophils migrate along a gradient of chemotactic factors immobilized by binding to negatively charged glycosaminoglycans (GAGs) and heparan sulfate proteoglycans (HSPGs) on the luminal surface of endothelial cells. PMN rolling and the engagement of chemokine receptors induces conformational changes in cell surface β2-integrins, which are transmembrane receptors that facilitate cell-extracellular matrix adhesion, allowing PMNs to bind with higher affinity to their ligands leading to a firm adhesion. During this process PMNs self-organize with a leading pseudopod and a trailing uropod, which subsequently leads to crawling, a process which is distinct from firm adhesion. The PMN crawling occurs unrelated to the direction of the blood flow and aims to find endothelial junctions for initiation of transmigration. PMNs may also facilitate transendothelial migration by production of reactive oxygen species (ROS). Transendothelial-migration is a critical step for the PMN to reach the site of inflammation and requires an activated state of the endothelium. At the site of inflammation, PMNs can elicit a number of immunological responses towards pathogens. These immune responses are tightly regulated in order to facilitate elimination of invading pathogens without inducing detrimental effects to host tissues. Specific activation/deactivation of PMN cell surface immune receptors by a wide range of extracellular signals regulates their effector functions. Bacteria can be engulfed and taken up by PMNs and degraded intracellularly in a process called phagocytosis. Intracellular vesicles and granules containing ROS and antibacterial proteins fuse with the phagosome creating a lysosome where the pathogen is degraded. Phagocytosis is mediated by antibodies or complement factors that bind to the surface of pathogens that are recognized by immune receptors on PMNs. These include Fcγ receptors which recognize the Fc-tail of the immunoglobulin G (IgG) which bounds pathogens via the IgG-Fab part. Complement is activated by antibodies bound to pathogens, primarily IgG and IgM, and subsequently cleaved complement products are deposited on pathogens and recognized by complement receptors (CRs) on PMNs. PMN granules with all their toxic components can also be released into the extracellular environment. PMNs may also respond to pathogens by releasing so-called neutrophil extracellular traps (NETs), which are formed by release of granule proteins and chromatin which together form extracellular fibers that trap and kill extracellular pathogens.

However, apart from the beneficial roles of PMNs in recruitment to sites of inflammation, recognition and phagocytosis of pathogens, production of ROS and secretion of NETs, PMNs may also have damaging functions.

According to a specific embodiment, the ARDS is associated with immune cells accumulation, such as macrophages, T cells, B cells, eosinophils and neutrophils (PMN) as discussed above.

According to a specific embodiment, the ARDS is associated with fibrosis. Detection of fibrosis is well known in the art such as by using imaging methods as X-ray, CT, MRI and the like.

As used herein the term “astrocyte” or “astrocytic cell” encompasses cells of the astrocyte lineage, i.e. glial progenitor cells, astrocyte precursor cells, and mature astrocytes, which for the purposes of the present invention can be isolated from native tissue, or can arise from a non-astrocytic cell by experimental manipulation, e.g., ex-vivo differentiated from stem cells. Astrocytes can be identified by markers specific for cells of the astrocyte lineage, e.g. GFAP, ALDH1L1, AQP4, EAAT-1 and 2, CD44, S100 beta and Vimentin. Astrocytes may have characteristics of functional astrocytes, that is, they may have the capacity of promoting synaptogenesis in primary neuronal cultures; of accumulating glycogen granules in processes; of phagocytosing synapses; immune modulation and the like. A “astrocyte precursor” or “astrocyte progenitor” is defined as a cell that is capable of giving rise to progeny that include astrocytes.

Astrocytes are the most numerous and diverse neuroglial cells in the CNS. An archetypal morphological feature of astrocytes is their expression of intermediate filaments, which form the cytoskeleton. The main types of astroglial intermediate filament proteins are glial fibrillary acidic protein (GFAP) and vimentin; expression of GFAP, ALDH1L1 and/or AQP4P are commonly used as a specific marker for the identification of astrocytes.

Astrocytes and other glial cells can release a variety of transmitters into the extracellular space, including glutamate, ATP, GABA and D-serine. Mechanisms of release may include: diffusion through high-permeability channels (e.g. volume-activated Cl-channels, unpaired connexin “hemichannels” or P2X7 pore-forming purinoceptors; through transporters, e.g. by reversal of excitatory amino acid transporters or exchange via the cystine-glutamate antiporter or organic anion transporters.

Astrocytes release neurotrophic factors (NTFs) upon activation, such as but not limited to VEGF, BDNF, GDNF, IGF-1, VEGF, NGF, MIF, TIMP-1, TIMP-2, MIDKINE and OPN.

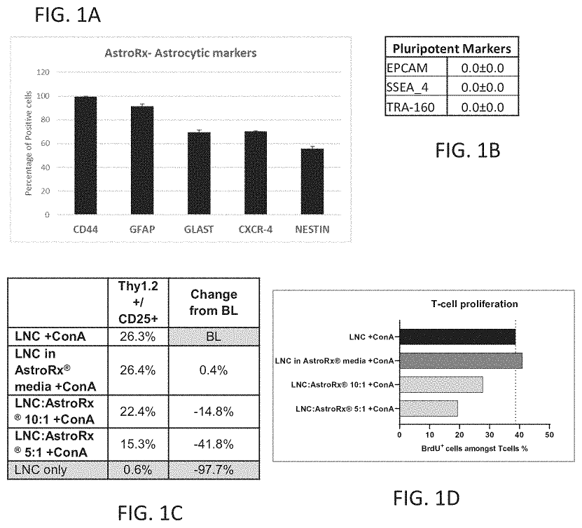

For example, the cells can be activated with LPS or IFNγ, as described in WO2015/049677. In addition, Immunomodulatory capacity of human astrocytes (AstroRx) tested in-vitro by mixed lymphocyte reaction test (FIGS. 1A-D) demonstrated that induced Concanavalin A (ConA) T cell proliferation was significantly decreased (Thy1.2+/CD25+ and CD3+/BrdU+) by addition of human astrocytes.

For secreted NTFs, supernatant is collected from an astrocytic culture. The amount of NTFs such as Glial Derived Neurotrophic Factor, (GDNF) or Brain Derived Neurotrophic Factor (BDNF) in the cell's culture supernatants may be quantified by using a GDNF or BDNF ELISA assay (GDNF G7620; BDNF G7610; Promega) according to the manufacturer's protocol, for example and without limitation. The amount of IGF-1 can be quantified using an IGF ELISA assay (IGF-1 DG100 R&D System), for example and without limitation. The amount of NGF can be quantified using an NGF ELISA assay (NGF Promega G7630), for example and without limitation.

The amount of VEGF can be quantified using a VEGF ELISA assay (DVE00 R&D systems) for example and without limitation.

According to a specific embodiment, the astrocytes are purified from a tissue, e.g., human brain tissue.

For example, and without limitation, US20160340648 teaches isolation of astrocytes from a human brain tissue.

Briefly, in some embodiments, the astrocytes are derived from tissue samples, e.g. brain tissue from one or more of cerebral cortex, cerebellum, hippocampus, mesencephalon, striatum, retina; etc., for example a biopsy specimen, etc., where the donor may be fetal, neonate, post-natal, adult, etc. In some such embodiments, the tissue is dissociated with high concentrations of papain, e.g. at least about 1 U/ml, at least about 5 U/ml, at least about 10 U/ml, or more. The tissue may be dissociated in papain for extended periods of time, e.g. at least about 30 minutes, at least about 45 minutes, at least about 60 minutes, or longer. In other embodiments a complex cell population comprising astrocytes is generated by in vitro differentiation.

For positive or negative selection, separation of the subject cell population utilizes affinity separation to provide a substantially pure population (e.g., above 50% of the cells in the preparation are astrocytes e.g., above 60%, 70%, 80%, 90% or substantially 100% pure). Techniques for affinity separation may include magnetic separation using antibody-coated magnetic beads, selective adhesion technique, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Any technique may be employed which is not unduly detrimental to the viability of the cells.

Positive immunoselection utilizes a reagent that selectively binds to HepaCam on the cells surface. Negative immunoselection is optionally performed to deplete cells of lineages other than astrocytes, e.g. to deplete myeloid cells; oligodendrocytes and oligodendrocyte precursor cells; neurons; endothelial cells; etc. In some embodiments, negative immunoselection is performed with reagents selective for one or more of CD45; GalC, 04, Thy1 and Banderiaea simplicifolia lectin 1 (BSL-1). In some embodiments two, three, four, five or more negative immunoselection reagents are used, e.g. in a cocktail or in separate negative selections. In some embodiments, a lineage cocktail comprising reagents for negative selection of each of myeloid cells; oligodendrocytes and oligodendrocyte precursor cells; neurons; endothelial cells. Where negative separation is used, it is usually performed prior to the positive selection, in order to deplete the cell population of undesirable cells. A positive selection is then performed.

According to a specific embodiment, the astrocytes are genetically modified.

For example, and without limitation, Park et al. [2009 Exp. Mol. Med. 41(7):487-500] teach human neuroprogenitor cells (hNPCs) having been genetically modified to express neurotrophic factors such as BDNF, VEGF, NT3, GDNF and VEGF using viral vectors (e.g., Lentivirus or Adenovirus).

The neural progenitor cells can be obtained from human fetal cadavers e.g., 13 weeks of gestation from which telencephalic brain tissue is dissected and dissociated using trypsin to generate a culture hNPCs.

According to a specific embodiment, the astrocytes are non-genetically modified.

According to a specific embodiment, the astrocytes have been ex vivo differentiated from stem cells. Hence, according to some embodiments, the cells are undergoing a step of ex vivo differentiation as a step of the present teachings.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, within the first couple of cell divisions after fertilization are the only cells that can differentiate into embryonic and extra-embryonic cells and are able to develop into a viable human being. Preferably, the phrase “pluripotent stem cells” refers to cells which can differentiate into all three embryonic germ layers, i.e., ectoderm, endoderm and mesoderm or remaining in an undifferentiated state. According to a specific embodiment, the cells are not totipotent stem cells. The pluripotent stem cells include embryonic stem cells (ESCs) and induced pluripotent stem cells (iPS). The multipotent stem cells include adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Placental and cord blood stem cells may also be referred to as “young stem cells”.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HADC100, HADC102, HADC106, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Embryonic stem cells (e.g., human ESCs) originating from an unfertilized ova stimulated by parthenogenesis (parthenotes) are known in the art (e.g., Zhenyu Lu et al., 2010. J. Assist Reprod. Genet. 27:285-291; “Derivation and long-term culture of human parthenogenetic embryonic stem cells using human foreskin feeders”, which is fully incorporated herein by reference). Parthenogenesis refers to the initiation of cell division by activation of ova in the absence of sperm cells, for example using electrical or chemical stimulation. The activated ovum (parthenote) is capable of developing into a primitive embryonic structure (called a blastocyst) but cannot develop to term as the cells are pluripotent, meaning that they cannot develop the necessary extra-embryonic tissues (such as amniotic fluid) needed for a viable human foetus.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); IH Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis.

Adult tissue stem cells can be isolated using various methods known in the art such as those disclosed by Alison, M. R. [J Pathol. 2003 200(5): 547-50], Cai, J. et al., [Blood Cells Mol Dis. 2003 31(1): 18-27], Collins, A. T. et al., [J Cell Sci. 2001; 114(Pt 21): 3865-72], Potten, C. S. and Morris, R. J. [Epithelial stem cells in vivo. 1988. J. Cell Sci. Suppl. 10, 45-62], Dominici, M et al., [J. Biol. Regul. Homeost. Agents. 2001, 15: 28-37], Caplan and Haynesworth [U.S. Pat. No. 5,486,359] Jones E. A. et al., [Arthritis Rheum. 2002, 46(12): 3349-60]. Fetal stem cells can be isolated using various methods known in the art such as those disclosed by Eventov-Friedman S, et al., PLoS Med. 2006, 3: e215; Eventov-Friedman S, et al., Proc Natl Acad Sci USA. 2005, 102: 2928-33; Dekel B, et al., 2003, Nat Med. 9: 53-60; and Dekel B, et al., 2002, J. Am. Soc. Nephrol. 13: 977-90. Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62]. Thus, an adult tissue such as, for example, prostate tissue is digested with Collagenase and subjected to repeated unit gravity centrifugation to separate the epithelial structures of the prostate (e.g., organoids, acini and ducts) from the stromal cells. Organoids are then disaggregated into single cell suspensions by incubation with Trypsin/EDTA (Life Technologies, Paisley, UK) and the basal, CD44-positive, stem cells are isolated from the luminal, CD57-positive, terminally differentiated secretory cells, using anti-human CD44 antibody (clone G44-26; Pharmingen, Becton Dickinson, Oxford, UK) labeling and incubation with MACS (Miltenyi Biotec Ltd, Surrey, UK) goat anti-mouse IgG microbeads. The cell suspension is then applied to a MACS column and the basal cells are eluted and re-suspended in WAJC 404 complete medium [Robinson, E. J. et al. (1998). Basal cells are progenitors of luminal cells in primary cultures of differentiating human prostatic epithelium Prostate 37, 149-160].

Since basal stem cells can adhere to basement membrane proteins more rapidly than other basal cells [Jones, P. H. et al. (1995). Stem cell patterning and fate in human epidermis. Cell 60, 83-93; Shinohara, T., et al. (1999). 01- and a6-integrin are surface markers on mouse spermatogonial stem cells. Proc. Natl. Acad. Sci. USA 96, 5504-5509] the CD44 positive basal cells are plated onto tissue culture dishes coated with either type I collagen (52 μg/ml), type IV collagen (88 μg/ml) or laminin 1 (100 μg/ml; Biocoat®, Becton Dickinson) previously blocked with 0.3% bovine serum albumin (fraction V, Sigma-Aldrich, Poole, UK) in Dulbecco's phosphate buffered saline (PBS; Oxoid Ltd, Basingstoke, UK). Following 5 minutes, the tissue culture dishes are washed with PBS and adherent cells, containing the prostate tissue basal stem cells are harvested with trypsin-EDTA.

The stem cells utilized by some embodiments of the invention can be BM-derived stem cells including stromal or mesenchymal stem cells (Dominici, M et al., 2001. Bone marrow mesenchymal cells: biological properties and clinical applications. J. Biol. Regul. Homeost. Agents. 15: 28-37). BM-derived stem cells may be obtained from iliac crest, femora, tibiae, spine, rib or other medullar spaces.

Of the above described BM-derived stem cells, mesenchymal stem cells are the formative pluripotent blast cells, and as such are preferred for use with some embodiments of the invention. Mesenchymal stem cells give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

It will be appreciated that undifferentiated stem cells are of a distinct morphology, which is clearly distinguishable from differentiated cells of embryo or adult origin by the skilled in the art. Typically, undifferentiated stem cells have high nuclear/cytoplasmic ratios, prominent nucleoli and compact colony formation with poorly discernable cell junctions.

Methods of obtaining mesenchymal stem cells which have been ex vivo differentiated to astrocytes are disclosed in WO 2014/024183, WO2006/134602 and WO2009/144718, the contents of each being incorporated herein by reference.

According to a specific embodiment, the astrocytes are ex vivo differentiated from embryonic stem cells (ESCs) or induced pluripotent stem cells (IPSCs).

According to a specific embodiment, ex vivo differentiation of astrocytes from pluripotent stem cells is effected as described in WO2015/049677.

Accordingly, the method is effected by:

(a) producing neurospheres (NS) from the pluripotent stem cells in a suspension culture in the presence of EGF and retinoic acid (RA);

(b) dissociating the NS so as to obtain astrocyte progenitor cells (APCs);

(c) expanding the APCs in an adherent culture in the presence of EGF and bFGF;

(d) differentiating the APCs to committed astrocytes in the presence of ascorbic acid.

As used herein “committed astrocytes”, refers to differentiated astrocytes exhibiting astrocyte functions but with reduced proliferative capabilities as compared to progenitor cells.

Specifically, a human ES cell line such as HADC100 is obtained (other examples are listed hereinabove).

Regardless of their origin, stem cells used in accordance with the present invention are at least 50% purified, 75% purified or at least 90% purified. When human embryonic stem cell lines are used, the human ES cell colonies are separated from their feeder layer (x-ray irradiated fibroblast-like cells) or matrix coated dish e.g., substrates (e.g. matrigel or an extracellular matrix component (e.g., collagen, laminin and fibronectin) or cationic adherent substrate (e.g. poly-D-lysine or Polyornithine with fibronectin (FN) or adherent substrates (e.g. matrigel or an extracellular matrix component (e.g., collagen, laminin and fibronectin) such as by mechanical and/or enzymatic means to provide substantially pure stem cell populations.

Once human stem cells arm obtained, they may be treated to differentiate into astrocytes. An exemplary method is described in the Materials and Methods section below, which is fully incorporated herein. It will be appreciated however, that the present teachings contemplates additional methods for producing astrocytes from pluripotent stem cells.

Examples of such differentiation protocols are known ion the art and include but not limited those described in:
Gupta, K.; Patani, R.; Baxter, P.; Serio, A.; Story, D.; Tsujita, T.; Hayes, J. D.; Pedersen, R. A.; Hardingham, G. E.; Chandran, S., Human embryonic stem cell derived astrocytes mediate non-cell-autonomous neuroprotection through endogenous and drug-induced mechanisms. Cell death and differentiation 2012, 19 (5), 779-87.
Juneja, D. S.; Nasuto, S.; Delivopoulos, E., Deriving Functional Astrocytes from Mouse Embryonic Stem Cells with a Fast and Efficient Protocol. Annu Int Conf IEEE Eng Med Biol Soc. 2994-2996, 2019, 2019
Byun, J. S.; Lee, C. O.; Oh, M.; Cha, D.; Kim, W. K.; Oh, K. J.; Bae, K. H.; Lee, S. C.; Han, B. S., Rapid differentiation of astrocytes from human embryonic stem cells. Neuroscience letters 2020, 716, 134681.

The culture medium used is selected according to the stem cell used. Thus, for example, a medium suitable for ES cell growth, can be, for example, DMEM/F12 (Sigma-Aldrich. St. Lewis, Mo.) or alpha MEM medium (Life Technologies Inc., Rockville, Md. USA), supplemented with supporting enzymes and hormones. These enzymes can be for example insulin (ActRapid; Novo Nordisk, Bagsvaerd, DENMARK), progesterone and/or Apo transferrin (Biological Industries, Beit Haemek. Israel). Other ingredients are listed in the Examples section. According to a specific embodiment, the medium is ITTSBP/B27 medium [ITTSPP/B27 is a mixture of DMEM/F12 containing 1% B27 supplement, 1% Glutamax, 1.5% Hepes at pH 7.4 (all from Thermo Scientific), 1% penicillin/streptomycin/amphotericin solution (Biological Industries), 25 μg/ml human insulin (ActRapid; Novo Nordisk), 50 μg/ml human Apo-transferrin (Athens), 6.3 ng/ml progesterone, 10 μg/ml putrescine, 50 ng/ml sodium selenite and 40 ng/ml triiodothyronine (T3) (all from Sigma)] supplemented with epidermal growth factor (EGF), 0.1-100 ng/ml. 1-100 ng/ml, 1-50 ng/ml, e.g., 20 ng/ml, e.g., such as the recombinant form of the protein available from R&D Systems.

The cells are cultured in this medium under non-adherent conditions (suspension culture) to generate neurospheres.

After 1-3 days in culture, the medium is supplemented with both EGF and retinoic acid.

As used herein the phrase “retinoic acid” refers to an active form (synthetic or natural) of vitamin A, capable of inducing neural cell differentiation. Examples of retinoic acid forms which can be used in accordance with the invention include, but are not limited to, retinoic acid, retinol, retinal, 11-cis-retinal, all-trans retinoic acid (ATRA), 13-cis retinoic acid and 9-cis-retinoic acid (all available at Sigma-Aldrich, St. Lewis, Mo.).

According to some embodiments, the retinoic acid is all-trans retinoic acid (ATRA).

In some embodiments of the invention, retinoic acid is used at a concentration range of 1-50 μM, 1-40 μM, 1-30 μM, e.g., 10 μM.

The cells are cultured under these conditions for 4-10 days e.g., 7 days. Medium may be changed periodically, e.g., daily.

In order to induce neurospheres (NS) ripening, retinoic acid is removed, and the medium is supplemented with EGF, e.g., same concentration range as above.

The culture time may last for 12-26 days (e.g., 18 days). Medium may be changed periodically, e.g., daily.

At this time ripened NS are obtained, these are typically distinguished by their round form and their color (yellowish).

As used herein the phrase “neurospheres” refers to quasi-spherical clusters or spheres containing mainly neural stem cells and early multipotent progenitors that can differentiate into neurons, oligodendrocytes and astrocytes as well as other glial cells. The cells are cultured until ripened neurospheres are formed.

As used herein the phrase “ripened neurospheres” refers to neurospheres in which some of the neural stem cells have differentiated to become specialized glial cells giving rise to astrocytes progenitors and oligodendrocyte progenitors having acquired glial markers (e.g. SOX10, Nk×2.2., NG2, A2B5, NESTIN, GFAP. CD44).

The ripened NS are transferred to adherent conditions (e.g., coated plates, as above), with a basic medium, e.g., is ITTSBP/B27 medium supplemented with epidermal growth factor (EGF), 0.1-100 ng/ml, 1-100 ng/ml, 1-50 ng/ml, e.g., 20 ng/ml e.g., such as the recombinant form of the protein available from R&D Systems, for 7-10 days. The medium may be changed periodically, e.g., every other day. This is considered passage 0.

The ripened neurospheres arm dissociated mechanically and/or with an enzyme (e.g., trypsin, e.g., TryPIE) and cultured in a culture medium e.g., N2/B27 supplemented with growth factors, which may be present at least in part of the culturing period to promote cell proliferation. According to an embodiment of this aspect of the present invention, such growth factors include for example. EGF (5-50 ng/ml) and bFGF (5-50 ng/ml) (R&D Systems).

The cells may be passaged periodically, e.g., every week. These cells are considered astrocyte progenitor cells (APCs), APC obtain early proliferative astrocytic markers such as Ki67, NESTIN, GFAP and S100β

Once sufficient cells are obtained, they may be frozen (in a cell bank) using cryopreserving agents such as DMSO in the presence of enriched solution such as human serum albumin or knock out serum replacement.

Alternatively, the astrocyte progenitor cells may be freshly used (without a step of freezing) in a further step of differentiation to committed astrocytes in the presence of ascorbic acid at a concentration from a range of 0.1 μg/ml to 1 mg/ml, 0.1-500 μg/ml, 0.1-300 μg/ml, 0.1-200 μg/ml, 0.1-100 μg/ml, 0.1-50 μg/ml, 1-500 μg/ml, 1-300 μg/ml, 1-200 μg/ml, 1-100 μg/ml, 1-50 μg/ml e.g., 50 μg/ml ascorbic acid (Sigma) and the culture is continued for 3-35 days e.g., 7 days to yield astrocytic cells that may be termed in some embodiments of the invention AstroRx™.

According to a specific embodiment, the cell preparation is subjected to a further step of purification which removes broken cells and any other cells derived particles such as exosomes, such that the cell preparation (composition) comprises viable cells in a predominant manner within the preparation.

Once cells are obtained they may be qualified for their ability to reduce pro-inflammatory cytokines (at the RNA and preferably protein level, e.g., by ELISA), reduce T cell proliferation (such as determined by MLR), and reduce neutrophil accumulation at the site of inflammation associated with ARDS.

As used herein “reduce” or “reduction” refers to at least 5%, 10%, 20%, 30%, 40%. 50%, 60%. 70%, 80% or more, say at least 90% reduction in the examined parameter or even complete abrogation as compared to control not treated with the astrocytes as described herein.

In any of the methods described herein, the cells can be administered either per se or, preferably as a part of a pharmaceutical composition that further comprises a pharmaceutically acceptable carrier.

As used herein a “pharmaceutical composition” refers to a preparation of cells described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol, DMSO, saline, emulsions and mixtures of organic solvents with water.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

According to a preferred embodiment of the present invention, the pharmaceutical carrier is an aqueous solution of saline.

Techniques for formulation and administration of drugs may be found in “Remington's Pharmaceutical Sciences,” Mack Publishing Co., Easton, Pa., latest edition, which is incorporated herein by reference.

Suitable routes of administration include direct administration into the tissue or organ of interest. Indirect administration can also be employed as long as the cells can reach the target site e.g., lungs.

Suitable routes of administration may, for example, include oral, rectal, transmucosal, especially transnasal, intestinal or parenteral delivery, including intramuscular, subcutaneous and intramedullary injections as well as intrathecal, intratracheal, direct intraventricular, intracardiac, e.g., into the right or left ventricular cavity, into the common coronary artery, intravenous, intraperitoneal, intranasal, or intraocular injections.

According to a specific embodiment, the administration is intravenous or intratracheal administration.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, a Coronavirus disease model such as that described in Kumar et al. VirusDis. (October-December 2020) 31(4):453-4. Non-limiting examples are discussed infra. Thus, one of the best mouse models used for COVID-19 is the K18-hACE2 transgenic mouse; Golden Syrian hamsters after infection with the SARS-CoV-2 virus showed weight loss and efficient viral replication in the nasal mucosa, and epithelial cells of the lower respiratory system; Ferrets have previously been used as a good model for the study of viral diseases especially for respiratory pathogens like influenza. For studying SARS-CoV-2 the animals were inoculated with the virus and found to develop similar symptoms as in humans within 2 and 12 days post-infection; Non-human primates can also be used, e.g., in particular M. mulatta (Rhesus macaques) can be a good model to study COVID-19 pathogenesis. NHP models have been developed for SARSCoV-2 to resemble the condition seen in human pathogenesis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1).

For injection, the active ingredients of the pharmaceutical composition may be formulated in aqueous solutions, preferably in physiologically compatible buffers such as PlasmaLyte, Hank's solution, Ringer's solution, or physiological salt buffer.

Dosage amount and interval may be adjusted individually to levels of the active ingredient which are sufficient to effectively regulate immunomodulator synthesis by the implanted cells. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated patient will be administered with an amount of cells which is sufficient to alleviate the symptoms of the disease, based on the monitoring indications.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Human Astrocytes Derived from Clinical Grade Human Embryonic Stem Cells (AstroRx®)

The protocol for manufacturing human astrocytes (AstroRx®) from embryonic stem cells was performed according to the protocol detailed in Izrael et al²⁶. In brief, hESC line HADC100, was used as starting material for the derivation of AstroRx cells. The hESCs were grown in feeder free Vitronectin coated flasks culture in Essential 8™ (E8) medium (Thermo Fischer Scientific). Once enough cells were obtained (10⁸-10¹⁰ hESCs), hESC were detached to generate neurospheres (NS) in suspension (3D) cultures. Harvested hESC aggregates were transferred into 100-mm ultralow attachment culture plates (Corning) containing ITTSPP/B27 medium. ITTSPP/B27 is a mixture of DMEM/F12 containing 1% B27 supplement, 1% Glutamax, 1.5% Hepes at pH 7.4 (all from Thermo Scientific), 1% penicillin/streptomycin/amphotericin solution (Biological Industries), 25 μg/ml human insulin (ActRapid; Novo Nordisk), 50 μg/ml human Apo-transferrin (Athens), 6.3 ng/ml progesterone, 10 μg/ml putrescine, 50 ng/ml sodium selenite and 40 ng/ml triiodothyronine (T3) (all from Sigma). ITTSPP/B27 was supplemented with 20 ng/ml r-human EGF (R&D Systems). After 2 days, the medium was replaced with ITTSPP/B27 supplemented with 20 ng/ml EGF and 10 μM ATRA (Sigma). The culture was continued in suspension in the nonadherent plates (ultralow adherence) or 7 days with daily replacement of the medium. During the last step, which allows for NS ripening, the culture was continued in ITTSPP/B27 medium supplemented with 20 ng/ml EGF for 18 days. Medium was replaced every other day. Then, round yellow NS were manually selected and transferred to GMP-compliant laminin 521 (from Biolamina) coated plates or flasks in ITTSPP/B27 supplemented with 20 ng/ml EGF. Medium was replaced every other day for 7-10 days (passage 0). To produce a monolayer of astrocyte progenitor cells (APC), the spheres were dissociated with TryplE (Thermo Scientific) to form a single cell layer culture and reseeded on laminin-521 coated flasks in “N2/B27 medium” composed of DMEM/F12 with 0.5% (v/v) N2 supplement, 1% (v/v) B27 supplement, 1% Glutamax and 1.5% Hepes at pH 7.4 (all from Thermo Scientific). The growth factors EGF and bFGF (R&D Systems) were added at 10 ng/ml each. The monolayer cells were further passaged weekly until enough cells were generated. Cells were then frozen in liquid nitrogen and stored as banks of APCs. Thawed APCs were seeded on Laminin-521 coated flasks and expanded with N2/B27 medium supplemented with EGF and bFGF. To allow APC differentiation toward committed astrocytes, growth factors (EGF and bFGF) were removed from the media and 50 μg/ml ascorbic acid (Sigma) was added and the culture was continued for 7 days to yield AstroRx cells.

Mixed lymphocyte reaction (MLR) test: 3 female C57BL mice at age of 6-8 weeks were euthanized by injection of a lethal dose of Pental (Pentobarbital Sodium). Lymph nodes were excised and drained to obtain lymph-node cells (LNC). LNC from all mice were pooled and then counted by a hemocytometer using trypan blue dye to exclude dead cells. One million LNC were seeded in RPMI-1640 medium (Biological Industries, 01-100-1A) supplemented with 2.5% fetal calf serum (Biological Industries, 04-121-1A), 1 mM L-glutamine (Biological Industries, 03-020-1C) and Penicillin-Streptomycin (Biological Industries, 03-031-1B). LNC cultures under multiple experimental conditions were maintained in flat-bottom plates, in duplicates, in a humidified atmosphere of 5% carbon dioxide at 37° C. Cells were harvested and assessed by flow cytometry for T cell amount and proliferation: Analysis of cells that express both T-cell markers; Thy1.2 and CD25 determined the percentage of T cells in LNC culture, 24 hours after activation with ConA.

Analysis of cells that express both CD3 (T-cell markers) and BrdU (proliferation marker) determined the percentage of proliferating T cells in LNC culture, 48 hours after activation with ConA. Testing was performed in several conditions (as shown in FIGS. 1A-D): A. T cell proliferation in the LNC culture for that aim LNC were kept in culture with and without 2.5 μg/mL ConA (Sigma, C5275). The percentage of T cells (Thy1.2+/CD25+) and proliferating T cells (CD3+/BrdU+) of the LNC cultures in the presence of ConA were set as baselines for T cell activation in culture (without immunomodulation). B. T cell proliferation in the presence of AstroRx® cells for that aim, AstroRx® cells were added to the LNC culture at ratios of 5:1 and 10:1 LNC:AstroRx®, with and without 2.5 μg/mL ConA and C. T cell proliferation in the presence of AstroRx® conditioned medium, for that aim, LNC in 0.5 ml RPMI-1640 were cultured with 0.5 ml conditioned medium with and without 2.5 μg/mL ConA.

Flow cytometry: Cells were analyzed by flow cytometry for identity and purity markers using the following antibodies: astrocytic markers; anti-GLAST (1:20; Miltenibiotec), anti-CD44 (1:20; BD Pharmingen), anti-CXCR4 (1:20; Biolegend), anti-GFAP (1:2000; Sigma), anti-Nestin (1:500; BD Pharmingen) and anti-AQP-4 (1:2000; Abcam). To detect any residual pluripotent cells in culture, the following pluripotent stem cells antibodies were used: anti-TRA-1-60 (1:50; Biolegend), anti-EPCAM (1:50; Biolegend), anti-SSEA4 (1:50; Biolegend). The Flow Cytometer FACS Canto II (BD) was operated with FACSDIVA software (BD). At least 10,000 events were collected per sample. For immune cells identity, the following antibodies were used FITC Rat Anti-Mouse I-A/I-E Clone 2G9 (RUO) (BD 553623), PerCP-Cy™5.5 Hamster Anti-Mouse CD3e (BD 551163), PerCP-Cy™5.5 Rat Anti-Mouse CD45R/B220 (552771), Mouse CCR3 PE-conjugated Antibody (R&D FAB729P) and APC-anti-mouse CD11 (Biolegend BLG-117310d).

Study design: A total of 30 BALB/C female mice were anesthetized using isoflurane and treated through the intratracheal route of administration with LPS (IT, 800 μg of LPS-ChemCruz, 055:B5) to induce ARDS. The animals were randomized to receive 200K, 500K AstroRx cells or sham control group injected with PlasmaLyte (Vehicle)(n=10 mice per experimental arm). Naive mice (n=4, without LPS instillation) were used as a healthy control. Treated animals received i.v. injection of AstroRx cells or PlasmaLyte 6 hr after LPS-induction. All animals were sacrificed 72 h after the LPS instillation. Measurements of survival, body weight, hematology, lung histopathology, flow cytometry analysis of immune cells as well as cytokine and chemokines were collected from bronchoalveolar lavage fluid (BALF) and serum.

Animal procedures: Female, 8 weeks old, BALB/C mice were obtained from Envigo (Israel) and maintained in an SIA facility (Science in Action, Ness Ziona, Israel). Animal handling was performed according to guidelines of the National Institute of Health (NIH) and the Association for Assessment and Accreditation of Laboratory Animal Care (AAALAC). The experiment was performed under the approval of “The Israel Board for Animal Experiments”. Bronchoalveolar lavage fluid (BALF) was collected by intratracheal injection of 0.5 ml PBS with 0.1 mM EDTA followed by gentle aspiration for 3 times. Recovered fluid was pooled and centrifuged. The BALF supernatant was preserved for the measurement of cytokines and chemokines. The sediment cells were resuspended and subjected to Flow cytometry analysis.

Histology: Lungs were harvested and fixed in 4% formaldehyde. The tissues were then trimmed in a standard position and put in embedding cassettes. One cassette was prepared per animal. Paraffin blocks were sectioned at ˜4 μm thickness, put on glass slides, and stained with hematoxylin and eosin (H&E). Pictures were taken using an Olympus microscope (BX60, serial NO. 7D04032) at objective magnification of ×4 and ×10 and microscope's Camera (Olympus DP73, serial NO. OH05504). A quantitative analysis for acute lung injury (ALI) was performed using a severity scoring scale of 0-2, based on the American Thoracic Society Documents, 2011²⁹. Analysis was performed by a certified veterinarian pathologist (Patho-logica Ltd., Ness Ziona, Israel) who was blinded to experimental treatment.

Neutrophils: Not visible within the field—a score of 0; 1-5 neutrophils—1; more than 5 neutrophils—2.
Fibrin: Not visible within the field—a score of 0; a single well-formed band of fibrin within the airspace—1; multiple eosinophilic membranes—2.
Thickened alveolar walls: Due to technical artifacts, only septal thickening that is equal or greater than twice normal was considered. Less than ×2—score 0; ×2-×4—score 1; more than ×4—score 2.

The analysis was based on measurements of 20 fields, using objective magnification of ×4 and ×10 (HPF).

Neutrophil cell count was performed using MATLAB color-based, brightness-based, and morphological-based segmentation. The cells were counted from a rectangle of 88,892 μm².

Cytokine and chemokine multiplex measurements: BALF cytokine concentrations were measured using ProcartaPlex Luminex platform (ThermoFischer, USA). The measurements were performed in duplicates (25 μl each) with a custom multiplex panel detecting the following mouse cytokines: IFNγ, TNFα, RANTES, IL-6, IL-10, IL-1α, IL-1β, IP-10, MIP1α, and MCP-1. Measurements were performed using Luminex MAGPIX instrument, and results were analyzed with Xponent 4.2 software according to manufacturer instructions.

Statistical analyses: Statistical analyses were performed using GraphPad Prism 7 software (GraphPad Software, San Diego, Calif.). For analysis of cytokine concentrations, TAT and tissue factor ELISAs, and neutrophil count, one-way ANOVA followed by Tukey's post hoc were performed. Histological scorings were analyzed using Kruskal-Wallis followed by Dunn's post hoc.

### Example 1

### Suppression of T Cell Proliferation by AstroRx® Cells

The immunomodulatory capacity of ex-vivo differentiated human astrocytes derived from embryonic stem cells, termed herein as AstroRx®, is not fully defined. For that aim, human astrocytes (AstroRx) expressing high levels of astrocytic markers (FIG. 1A), and no expression of pluripotent stem cells markers (FIG. 1B), were used for the in-vitro and in-vivo studies. The percentage of Thy1.2+/CD25+ cells in a culture of lymph node cells (LNC) with a T cell mitogen Concanavalin A (ConA) was chosen to be the base line (BL) for the estimation of percentage of T cells in the culture. When ConA was not added to the culture, the percentage of T cells in the culture was below 1%. Addition of either AstroRx® cells or AstroRx® cells' conditioned medium did not affect the percentage of T cells in the culture (FIG. 1C).

In contrast, in the presence of ConA, AstroRx® cells profoundly influenced T cell population (Thy1.2+/CD25+), which decreased by about 15% in the LNC:AstroRx® 10:1 culture. A greater inhibitory effect was observed when using higher ratio of AstroRx® to LNC, (−)42% in LNC:AstroRx® 5:1 cultures. This effect was not observed when AstroRx*-conditioned medium was added (FIG. 1C).

The percentage of CD3+/BrdU+ cells in a culture of LNC with ConA was chosen to be the base line (BL) for the estimation of percentage of proliferating T cells in the culture. When ConA was not added to the culture, the percentage of human proliferating T cells in the culture was below 1%, and for mNPC was low as well (2%). Addition of different ratios of AstroRx® cells to LNCs did not affect the percentage of proliferating T cells in the culture (not shown). In contrast, in the presence of ConA, AstroRx® cells profoundly influenced proliferating T cell population (CD3+/BrdU+), which decreased by about 28% in the LNC:AstroRx® 10:1 culture. Higher ratios of AstroRx® to LNC resulted in a greater inhibitory effect by AstroRx® cells, about (−)49% and (−)43% in LNC:AstroRx® 5:1 and LNC:AstroRx® 2:1 cultures, respectively (FIG. 1D).

This data suggests immunomodulatory properties exerted by AstroRx® cells. The inhibitory effect on T cell proliferation by AstroRx cells was not demonstrated by AstroRx®-conditioned medium. The requirement of AstoRx® cell presence for their immunomodulatory activities suggests, without being bound by theory, that this activity is mediated by a direct contact or communication between AstroRx® cells and cells in the LNC culture.

### Example 2

### AstroRx Cells Alleviate LPS-Induced Pulmonary Inflammation and Fibrosis

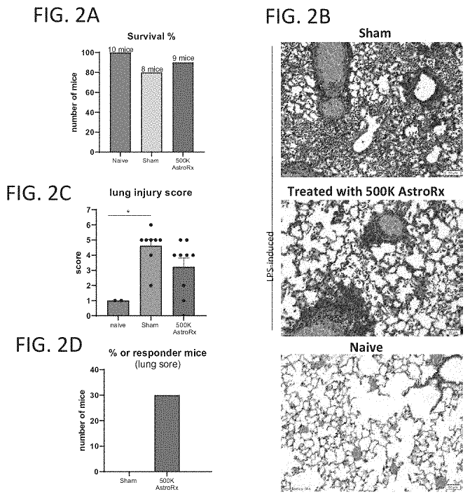

Administration of lipopolysaccharide (LPS) to mice induces severe lung damage and is a prevalent ARDS animal model²⁸. To assess their physiological effects, AstroRx cells were injected i.v. after LPS-induction. The survival of LPS-induced mice treated with AstroRx at both cell concentration was higher compared to sham injected animals (FIG. 2A). Histological analysis of lung sections showed a significant lung damage 72 h after LPS treatment (FIG. 2B). Treatment with AstroRx cells at a concentration of 500,000 cells alleviated the LPS-induced physical damage (FIG. 2B). A quantitative analysis for Acute Lung Injury (ALI) was performed using a severity scoring scale of 0-2, based on the American Thoracic Society Documents²⁹ assessing alveolar wall thickness, fibrin presence, and neutrophil accumulation which sums together to a total severity score. Treatment with AstroRx cells significantly lowered the total severity score as compared to sham injected LPS—induced animals, 3.22 compared to 4.6 (FIG. 2C). According to lung score the animals were categorized into responders (lung score ≤2) and non-responders (lung score>2). The percentage of responders was higher in AstroRx treated mice compared to sham injected mice (FIG. 2D).

### Example 3

### AstroRx Attenuates LPS-Induced Cytokine and Chemokine Storm in BALF and Serum

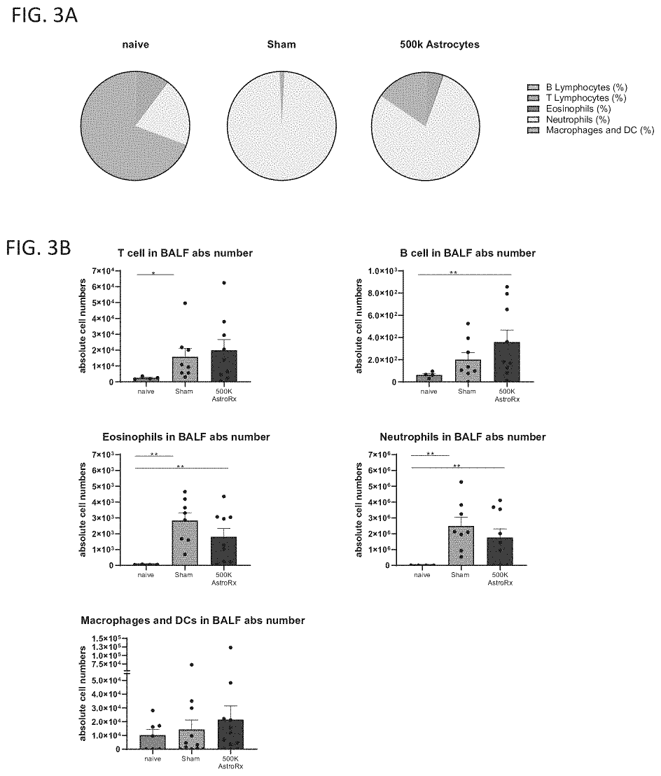

To understand the factors that contributed to reduced lung damage following AstroRx treatment, the present inventors analyzed the absolute number and proportion of B and T lymphocytes, Eosinophils, Neutrophils and Macrophages immune cells in bronchoalveolar lavage fluid (BALF). The proportion of immune cells were modulated following AstroRx (500K) treatment compared to sham injected (FIG. 3A). The absolute number of each cell type was analyzed. A significant reduction of number Neutrophils was observed in AstroRx (500,000) treated LPS-induced mice compared to sham injected animals (FIG. 3B).

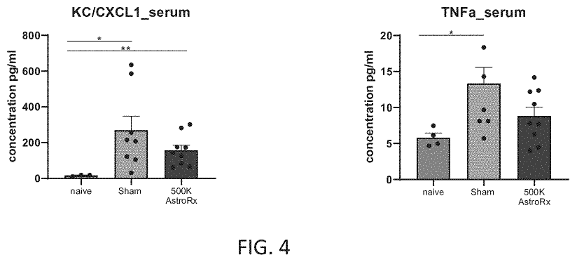

Next, the effect of AstroRx on cytokines and chemokines secretion was assessed in the serum. LPS induced Cytokine storm as shown by elevated levels of (interleukin (IL)-1β, IL-5, IL-6, and tumor necrosis factor (TNF)) and chemokines (C—C motif chemokine ligand (CCL)-2, and CCL-1) in sham injected animals compared to healthy mice. AstroRx (500,00) treated animals presented lower levels of CXCL1 and TNFα in the serum as shown in FIG. 4.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Included in the Document

1. Sweeney, M. D.; Zhao, Z.; Montagne, A.; Nelson, A. R.; Zlokovic, B. V., Blood-Brain Barrier: From Physiology to Disease and Back. Physiol Rev 2019, 99 (1), 21-78.
2. Lin, S. H.; Zhao, Y. S.; Zhou, D. X.; Zhou, F. C.; Xu, F., Coronavirus disease 2019 (COVID-19): cytokine storms, hyper-inflammatory phenotypes, and acute respiratory distress syndrome. Genes Dis 2020, 7 (4), 520-527.
3. Jiang, F.; Deng, L.; Zhang, L.; Cai, Y.; Cheung, C. W.; Xia, Z., Review of the Clinical Characteristics of Coronavirus Disease 2019 (COVID-19). J Gen Intern Med 2020, 35 (5), 1545-1549.
4. Zhou, F.; Yu, T.; Du, R.; Fan, G.; Liu, Y.; Liu, Z.; Xiang, J.; Wang, Y.; Song, B.; Gu, X.; Guan, L.; Wei, Y.; Li, H.; Wu, X.; Xu, J.; Tu, S.; Zhang, Y.; Chen, H.; Cao, B., Clinical course and risk factors for mortality of adult inpatients with COVID-19 in Wuhan, China: a retrospective cohort study. Lancet 2020, 395 (10229), 1054-1062.
5. Carver, C.; Jones, N., Cardiac injury, ARDS and COVID-19 meta-analysis validity—Correspondence in response to Santoso et al. Am J Emerg Med 2020.
6. Lian, J.; Jin, X.; Hao, S.; Cai, H.; Zhang, S.; Zheng, L.; Jia, H.; Hu, J.; Gao, J.; Zhang, Y.; Zhang, X.; Yu, G.; Wang, X.; Gu, J.; Ye, C.; Jin, C.; Lu, Y.; Yu, X.; Yu, X.; Ren, Y.; Qiu, Y.; Li, L.; Sheng, J.; Yang, Y., Analysis of Epidemiological and Clinical Features in Older Patients With Coronavirus Disease 2019 (COVID-19) Outside Wuhan. Clin Infect Dis 2020, 71 (15), 740-747.
7. Rocco, P. R.; Dos Santos, C.; Pelosi, P., Lung parenchyma remodeling in acute respiratory distress syndrome. Minerva Anestesiol 2009, 75 (12), 730-40.
8. Thille, A. W.; Esteban, A.; Fernandez-Segoviano, P.; Rodriguez, J. M.; Aramburu, J. A.; Vargas-Errazuriz, P.; Martin-Pellicer, A.; Lorente, J. A.; Frutos-Vivar, F., Chronology of histological lesions in acute respiratory distress syndrome with diffuse alveolar damage: a prospective cohort study of clinical autopsies. Lancet Respir Med 2013, 1 (5), 395-401.
9. Meduri, G. U.; Eltorky, M. A., Understanding ARDS-associated fibroproliferation. Intensive Care Med 2015, 41 (3), 517-20.
10. Akbari, H.; Tabrizi, R.; Lankarani, K. B.; Aria, H.; Vakili, S.; Asadian, F.; Noroozi, S.; Keshavarz, P.; Faramarz, S., The role of cytokine profile and lymphocyte subsets in the severity of coronavirus disease 2019 (COVID-19): A systematic review and meta-analysis. Life sciences 2020, 258, 118167.
11. Uckun, F. M., Reducing the Fatality Rate of COVID-19 by Applying Clinical Insights From Immuno-Oncology and Lung Transplantation. Front Pharmacol 2020, 11, 796.
12. Kempuraj, D.; Selvakumar, G. P.; Ahmed, M. E.; Raikwar, S. P.; Thangavel, R.; Khan, A.; Zaheer, S. A.; Iyer, S. S.; Burton, C.; James, D.; Zaheer, A., COVID-19, Mast Cells, Cytokine Storm, Psychological Stress, and Neuroinflammation. Neuroscientist 2020, 26 (5-6), 402-414.
13. Pelaia, C.; Tinello, C.; Vatrella, A.; De Sarro, G.; Pelaia, G., Lung under attack by COVID-19-induced cytokine storm: pathogenic mechanisms and therapeutic implications. Ther Adv Respir Dis 2020, 14, 1753466620933508.
14. Allen, N. J., Astrocyte regulation of synaptic behavior. Annu Rev Cell Dev Biol 2014, 30, 439-63.
15. Allen, N. J.; Eroglu, C., Cell Biology of Astrocyte-Synapse Interactions. Neuron 2017, 96 (3), 697-708.
16. Izrael, M.; Slutsky, S. G.; Revel, M., Rising Stars: Astrocytes as a Therapeutic Target for ALS Disease. Front Neurosci 2020, 14, 824.
17. Fasciani, I.; Pluta, P.; Gonzalez-Nieto, D.; Martinez-Montero, P.; Molano, J.; Paino, C. L.; Millet, O.; Barrio, L. C., Directional coupling of oligodendrocyte connexin-47 and astrocyte connexin-43 gap junctions. Glia 2018, 66 (11), 2340-2352.
18. Liddelow, S. A.; Guttenplan, K. A.; Clarke, L. E.; Bennett, F. C.; Bohlen, C. J.; Schirmer, L.; Bennett, M. L.; Munch, A. E.; Chung, W. S.; Peterson, T. C.; Wilton, D. K.; Frouin, A.; Napier, B. A.; Panicker, N.; Kumar, M.; Buckwalter, M. S.; Rowitch, D. H.; Dawson, V. L.; Dawson, T. M.; Stevens, B.; Barres, B. A., Neurotoxic reactive astrocytes are induced by activated microglia. Nature 2017, 541 (7638), 481-487.
19. Liddelow, S. A.; Barres, B. A., Reactive Astrocytes: Production, Function, and Therapeutic Potential. Immunity 2017, 46 (6), 957-967.
20. Farina, C.; Aloisi, F.; Meinl, E., Astrocytes are active players in cerebral innate immunity. Trends Immunol 2007, 28 (3), 138-45.
21. Pekny, M.; Pekna, M.; Messing, A.; Steinhauser, C.; Lee, J. M.; Parpura, V.; Hol, E. M.; Sofroniew, M. V.; Verkhratsky, A., Astrocytes: a central element in neurological diseases. Acta neuropathologica 2016, 131 (3), 323-45.
22. Sofroniew, M. V., Astrogliosis. Cold Spring Harb Perspect Biol 2014, 7(2), a020420.
23. Sofroniew, M. V., Multiple roles for astrocytes as effectors of cytokines and inflammatory mediators. Neuroscientist 2014, 20 (2), 160-72.
24. Owens, T.; Bechmann, I.; Engelhardt, B., Perivascular spaces and the two steps to neuroinflammation. Journal of neuropathology and experimental neurology 2008, 67(12), 1113-21.
25. Wilson, E. H.; Weninger, W.; Hunter, C. A., Trafficking of immune cells in the central nervous system. The Journal of clinical investigation 2010, 120 (5), 1368-79.
26. Izrael, M.; Slutsky, S. G.; Admoni, T.; Cohen, L.; Granit, A.; Hasson, A.; Itskovitz-Eldor, J.; Krush Paker, L.; Kuperstein, G.; Lavon, N.; Yehezkel Ionescu, S.; Solmesky, L. J.; Zaguri, R.; Zhuravlev, A.; Volman, E.; Chebath, J.; Revel, M., Safety and efficacy of human embryonic stem cell-derived astrocytes following intrathecal transplantation in SOD1(G93A) and NSG animal models. Stem cell research & therapy 2018, 9 (1), 152.
27. Einstein, O.; Fainstein, N.; Vaknin, I.; Mizrachi-Kol, R.; Reihartz, E.; Grigoriadis, N.; Lavon, I.; Baniyash, M.; Lassmann, H.; Ben-Hur, T., Neural precursors attenuate autoimmune encephalomyelitis by peripheral immunosuppression. Annals of neurology 2007, 61 (3), 209-18.
28. Matute-Bello, G.; Frevert, C. W.; Martin, T. R., Animal models of acute lung injury. Am J Physiol Lung Cell Mol Physiol 2008, 295 (3), L379-99.
29. Matute-Bello, G.; Downey, G.; Moore, B. B.; Groshong, S. D.; Matthay, M. A.; Slutsky, A. S.; Kuebler, W. M.; Acute Lung Injury in Animals Study, G., An official American Thoracic Society workshop report: features and measurements of experimental acute lung injury in animals. Am J Respir Cell Mol Biol 2011, 44 (5), 725-38.
30. Kuhle, J.; Lindberg, R. L.; Regeniter, A.; Mehling, M.; Steck, A. J.; Kappos, L.; Czaplinski, A., Increased levels of inflammatory chemokines in amyotrophic lateral sclerosis. Eur J Neurol 2009, 16 (6), 771-4.
31. Fiala, M.; Chattopadhay, M.; La Cava, A.; Tse, E.; Liu, G.; Lourenco, E.; Eskin, A.; Liu, P. T.; Magpantay, L.; Tse, S.; Mahanian, M.; Weitzman, R.; Tong, J.; Nguyen, C.; Cho, T.; Koo, P.; Sayre, J.; Martinez-Maza, O.; Rosenthal, M. J.; Wiedau-Pazos, M., IL-17A is increased in the serum and in spinal cord CD8 and mast cells of ALS patients. Journal of neuroinflammation 2010, 7, 76.
32. Moreau, C.; Devos, D.; Brunaud-Danel, V.; Defebvre, L.; Perez, T.; Destee, A.; Tonnel, A. B.; Lassalle, P.; Just, N., Elevated IL-6 and TNF-alpha levels in patients with ALS: inflammation or hypoxia? Neurology 2005, 65 (12), 1958-60.
33. Zhang, R.; Miller, R. G.; Gascon, R.; Champion, S.; Katz, J.; Lancero, M.; Narvaez, A.; Honrada, R.; Ruvalcaba, D.; McGrath, M. S., Circulating endotoxin and systemic immune activation in sporadic amyotrophic lateral sclerosis (sALS). Journal of neuroimmunology 2009, 206 (1-2), 121-4.

What is claimed is:

1. A method of treating acute respiratory distress syndrome (ARDS) in a subject in need thereof, the method comprising administering to the subject a composition comprising a therapeutically effective amount of astrocytes, thereby treating the ARDS.

2. The method of claim 1, wherein said astrocytes having been ex vivo differentiated from stem cells.

3. The method of claim 2, wherein said stem cells are pluripotent stem cells.

4. The method of claim 3, wherein said pluripotent stem cells are embryonic stem cells.

5. The method of claim 3, wherein said pluripotent stem cells are induced pluripotent stem (IPS) cells.

6. The method of claim 2, wherein said stem cells are mesenchymal stem cells.

7. The method of claim 2, further comprising ex vivo differentiating said astrocytes prior to said administering.

8. The method of claim 7, wherein said ex vivo differentiating said astrocytes from said pluripotent stem cells comprise:
(a) producing neurospheres (NS) from the pluripotent stem cells in a suspension culture in the presence of EGF and retinoic acid;
(b) dissociating the NS so as to obtain astrocyte progenitor cells (APCs);
(c) expanding said APCs in an adherent culture in the presence of EGF and bFGF;
(d) differentiating said APCs to committed astrocytes in the presence of ascorbic acid.

9. The method of claim 1, wherein said astrocytes are formulated for intravenous or intratracheal administration.

10. The method of claim 1, wherein said ARDS is associated with virus infection.

11. The method of claim 10, wherein said virus is a Coronavirus.

12. The method of claim 11, wherein said Coronavirus is SARS-CoV-2.

13. The method of claim 1, wherein said astrocytes reduce neutrophil accumulation at the site of inflammation associated with the ARDS.

14. The method of claim 1, wherein said astrocytes reduce pro-inflammatory cytokines.

15. The method of claim 14, wherein said pro-inflammatory cytokines are selected from the group consisting of TNF-alpha, ILb1 and IL-6.

16. The method of claim 1, wherein said astrocytes reduce T cells proliferation.

17. The method of claim 1, wherein said astrocytes reduce fibrosis.

18. The method of claim 1, wherein said composition comprises a viable form of said astrocytes in predominant manner.
